(19) United States
(12) Patent Application Publication
(10) Pub. No.: US 2007/0231906 A1
(43) Pub. Date: Oct. 4, 2007

(54) Three Frame cDNA Expression Libraries for Functional Screening of Proteins

(75) Inventors: Pei-Zhong Tang, Carlsbad, CA (US); Phillip Gray, Carlsbad, CA (US)

Correspondence Address: INVITROGEN CORPORATION;C/O INTELLEVATE, P.O. BOX 52050, MINNEAPOLIS, MN, 55402, US

(73) Assignee: Invitrogen Corporation, Carlsbad, CA (US)

(21) Appl. No.: 11/552,505

(22) Filed: Oct. 24, 2006

Related U.S. Application Data
(60) Provisional application No. 60/729,829, filed on Oct. 25, 2005.

(51) Int. Cl. C12N 15/64; C07H 21/04; C12N 15/74; C12N 15/81; C12N 15/82; C12N 15/85

(52) U.S. Cl. 435/455; 435/320.1; 435/440; 435/468; 435/471; 536/23.1; 536/24.33

(57) ABSTRACT

Compositions and methods are provided for generating three frame cDNA expression libraries for functional screening of proteins. The invention includes sets of 5′ adapters for cloning cDNA molecules in which the sets include three adapters that can be used to clone a particular cDNA in all three reading frames. The libraries so generated have greater complexity of expressed open reading frames, and thus can improve the success of functional protein screens, such as two hybrid screens. The adapters also have recombination sites for efficient cloning and transfer between systems.

This application claims benefit of priority to U.S. Provisional application 60/729,879, filed Oct. 24, 2005, entitled “Three Frame cDNA Expression Libraries for Functional Screening of Proteins” naming Tang, Chappell, and Gray as inventors, which is herein incorporated by reference in its entirety.

### BACKGROUND OF THE INVENTION

1. Field of the Invention

The invention relates generally to the manufacture and use of cDNA expression libraries, and more specifically to compositions, systems, and methods for generating cDNA expression libraries for functional screens for protein activity, such as two hybrid assays that screen for interacting proteins.

2. Background Information

The yeast two-hybrid system is a powerful tool for identifying protein-protein interactions. The system is based on a split transcription factor, where proteins are expressed in S. cerevisiae as fusions to either the DNA binding domain (DBD) or transcriptional activator domain (AD). A positive protein-protein interaction reconstitutes a functional transcription factor, which is capable of activating reporter genes in genetically modified strains of S. cerevisiae (Fields and Song, Nature 340: 245-6 (1989)).

In performing screens for proteins that interact with a protein of interest, the protein of interest is expressed in a cell line or strain as a “bait” protein fused to either a DBD or AD. To find a “prey” protein that interacts with the bait, an expression cDNA library is constructed of RNA isolated from the cell type of interest, in an expression vector configured to express cDNAs of the library as fusions to an AD or DBD (whichever is not fused to the bait). To perform a comprehensive screen for positive interactors, it is critical that the library have as high a complexity as possible to maximize the chances of finding relevant prey proteins.

Reverse transcriptase, used to make cDNA, begins at the 3′ end of an RNA template and proceeds toward the 5′ end of the RNA template as it polymerizes cDNA. When the reverse transcriptase pauses or stops, first strand synthesis can also come to a halt. The second strand synthesis then begins at that 5′-most stopping point, which becomes the 5′ end of the synthesized cDNA. Several researchers (see, for example, Harrison et al. (1998) Nucleic Acids Research 26: 3433-3442; Klarmann et al. (1993) J. of Biol. Chem. 268: 9793-9802; DeStafano et al. (1991) J. of Biol. Chem. 266: 7423-7431) have found there is a bias in the pausing of reverse transcriptase enzymes. This pausing, which can prevent further 5′ synthesis and thus defines the 5′ end of the cDNA, can preferentially occur at particular base residues within the sequence.

When a synthesized cDNA is cloned into an expression vector so that it becomes the carboxy-terminal portion of a fusion protein, the 5′ end of the cDNA sets the reading frame that will be followed by the downstream cDNA sequences, based on the reading frame of the preceding sequences in the expression vector that the 5′ end of the cDNA becomes fused to. For example, in two hybrid systems, cDNAs are cloned as C-terminal fusions to either the AD or DBD, which is encoded by the vector. Thus, the protein-encoding reading frame of the cDNA may not be in register with the reading frame set by the N-terminal AD or DBD domain, depending on whether the 5′ end of the cDNA is a base pair in the first, second, or third position of a codon that is formed at the point at which the cDNA and expression vector are joined. Bias in the position of termination of first strand synthesis of some cDNA sequences during the manufacture of cDNA libraries can therefore lead to a lack of functional expression fusion constructs generated from those cDNAs in expression screening systems, regardless of the complexity of the cDNA library.

Another difficulty in obtaining representative expression libraries for protein functional screens is that 5′ UTRs of cloned cDNAs often include stop codons that can prevent expression of the cDNA when it is fused downstream from an open reading frame, as is the case for many yeast two hybrid assays.

There is a need for improved methods and systems for manufacture of cDNA expression libraries for use in functional screens, such that fully representational expression libraries are obtained despite the bias of reverse transcriptase pausing that establishes the 5′ end of synthesized cDNAs, and without 5′ UTR stop codons that can prevent expression of a cDNA as a fusion protein.

### SUMMARY OF THE INVENTION

The invention provides compositions and methods for protein functional screening using expression libraries that are not reading-frame biased. In particular the invention provides reagents, systems, and methods for constructing and performing two hybrid screens for interacting proteins, in which proteins expressed by a cell type of interest can be assayed by expressing cDNA synthesized from RNA isolated from the cells. The complexity of the expression library, and therefore the frequency of detecting positive interactors, is greatly enhanced using cDNAs made using the methods of the present invention. These methods include the use of a set of adapters that allow for cloning the cDNA in all three reading frames. In preferred embodiments, the adapters comprise recombination sites that allow for integrating the synthesized cDNA into vectors with high efficiency, and for transferring the cloned cDNAs from one vector to another with high efficiency such that a synthesized cDNA library can be transformed into bacteria for selection and optionally, amplification, and transferred into other cell types, such as eukaryotic cells for functional assays.

A first aspect of the invention is a set of three oligonucleotide adapters for cloning a nucleic acid fragment into an expression vector, in which each one of the set of adapters, when ligated to the same nucleic acid fragment, will cause the nucleic acid fragment, when cloned into an expression vector, to be in a different reading frame. Thus a set of three oligonucleotide adapters will allow cloning of a DNA fragment into an expression vector in all three reading frames. In preferred embodiments, the set of oligonucleotide adapters is used for cloning cDNA into an expression vector. Preferably, the adapters also include recognition sequences for at least one nucleic acid recombinase.

A second aspect of the invention is methods of making a cDNA expression library, in which the oligonucleotide adapters are ligated to cDNA, and the cDNA is inserted into an expression vector. The expression vector can be a vector of any type and can be designed for replication in any cell type. The insertion of cDNA into an expression vector can be direct or indirect. For example, cDNA can be introduced into an expression vector by means of one or more intermediate vectors.

A third aspect of the invention is a three reading frame cDNA library made using the methods of the present invention. The library can be in a vector designed for replication and/or expression in any cell type.

A fourth aspect of the present invention is a method of performing a screen for protein activity using a cDNA expression library of the present invention. The screen can be in vitro or in vivo and can be a screen for a protein activity or a cellular phenotype or activity.

A fifth aspect of the present invention is a method of performing a two hybrid screen for an interacting protein using a cDNA expression library of the present invention.

A sixth aspect of the present invention is kits for making three frame expression cDNA libraries. The kits include at least one set of three frame oligonucleotide adapters, and at least one of: a 3′ primer for first strand cDNA synthesis, a ligase, a topoisomerase, an enzyme formulation for nucleic acid recombination, or a vector for insertion of cDNA. The kit can also include buffers, test reagents, restriction enzymes, and one or more purification or separation reagents.

### BRIEF DESCRIPTION OF THE DRAWINGS

FIG. 1 is a map of expression vector pDEST22.

FIG. 2 is a map of expression vector pDEST32.

FIG. 3 is a flow chart for the use of three frame cDNA libraries in two hybrid protein interaction screens.

### DETAILED DESCRIPTION OF THE INVENTION

The present invention is based on the need to improve the efficiency of functional screens for proteins of interest that rely on expression cloning of a population of cDNA molecules. In exemplary embodiments, the proteins expressed from the cDNA population are surveyed for their activity. In these studies, it is important to have the highest possible representation of expressed proteins in the functional screen. The invention described herein provides compositions and methods for increasing the representation of expressed cDNAs by providing a set of three reading frame oligonucleotide adapters, in which the set comprises three adapters having sequences that facilitate cloning of the cDNA into a vector in all three reading frames. A set of three frame adapters is advantageously used to clone cDNAs, such that when each of the three adapters is independently joined with a given cDNA molecule, and the cDNA molecule is integrated into an expression vector, each of the three possible reading frames of the nucleic acid sequence is cloned in-frame with the expression sequences of the vector. This ensures that the reading frame or the cDNA that is the open reading frame of the protein encoded by the cDNA is represented as one of the three cloned reading frames. Thus, using the compositions and methods of the present invention, cloning of a nucleic acid such as a cDNA such that its open reading frame will be expressed by the vector does not rely on the random chance that the 5′-most nucleotide of the cDNA permits the open reading frame of the cDNA to be in frame with the expression vector sequences, which in exemplary embodiments include protein coding sequences of the vector.

Oligonucleotide Adapters

By “adapter” is meant an oligonucleotide that includes sequences that when joined to a nucleic acid molecule, facilitate joining of the nucleic acid molecule to another nucleic acid molecule, such as, for example, a vector. A sequence that facilitates joining of one nucleic acid molecule to another, can be, for example, a restriction enzyme site, a recombination site, or a topoisomerase recognition site, herein collectively referred to as “cloning sites”. Different adapters of the set can have the same or different cloning (or vector integration) sites. In a preferred embodiment an adapter set of oligonucleotide (“oligo”) adapters includes first adapter, a second adapter, and a third adapter, in which the three adapters have a common cloning site, one end of the adapters is designed to be ligated to a 5′ end of a cDNA molecule (the “ligation end” of the adapter), and the length of the three adapters from the cloning site to the ligation end of the adapter differs, such that each of the three adapters of the set, when ligated to the 5′ end of the same cDNA molecule, will place the cDNA sequence in a different reading frame of the same expression vector. For example, the lengths of the three oligo adapters from the integration site to the end of the adapter designed to be ligated to a cDNA can be ‘n’ for the first adapter, ‘n+(3)i+1’ for the second adapter, and ‘n+(3)i+2’ for the third adapter, where i is an integer (0, 1, 2, 3, . . . ) and is preferably 0, such that the lengths of the three oligo adapters from the integration site to the end of the adapter designed to be ligated to a cDNA is, in exemplary embodiments, ‘n’ for the first adapter, ‘n+1’ for the second adapter, and ‘n+2’ for the third adapter. The vector integration site can be any site that allows the adapter-joined cDNA to integrate into a vector, for example, a restriction enzyme site, a recombination site, or topoisomerase recognition site.

In preferred embodiments, the adapters of a set share the same sequence (are identical in sequence), with the exception that the three oligomer adapters differ in length, such that a first adapter of the set is, for example, x nucleotides long, a second nucleotide adapter is x+1 nucleotides long, and a third adapter of the set is x+2 nucleotides long.

The oligonucleotide adapters are substantially double stranded when used in cloning reactions, except that one end of the adapters, hereinafter to be referred to as nonligating end, comprises a single-stranded overhang that is at least one nucleotide in length, for example, from one to twenty nucleotides in length, and preferably from two to ten nucleotides in length. This single-stranded region of the adapter ensures that this end of the adapter will not ligate to a cDNA molecule during the cloning process, but rather will remain a free end after a ligation reaction. The overhang can be a 3′ or a 5′ overhang. In illustrative embodiments, the single-stranded overhang on the nonligating end of the adapter can be a 5′ overhang of about 3, 4, 5, 6, or 7 nucleotides in length.

Oligonucleotide adapters that are substantially double-stranded when ligated to a cDNA or used to clone cDNA into an expression vector can be provided in single stranded form. For example, an oligonucleotide adapter can be provided as a “long strand” that has the overhang sequences that will remain single-stranded in cloning reactions, and a “short strand” which is complementary to substantially all of the sequence of the long strand, excepting the overhang sequence of the long strand. The long and short strands can be annealed prior to use of the adapter in a ligation reaction, for example by heating and then gradually cooling the two strands in the same solution.

The end of the adapter that is opposite to the nonligating end is herein termed the ligating end of the adapter. In some preferred embodiments, the three adapters are identical in sequence, with the exception that a second adapter has one more base pair at the end ligating end than a first adapter of the set, and a third adapter of the set has two more base pairs at the ligating end than a second adapter of the set. The oligomeric adapters can be of any length, for example from about twelve to about 100 base pairs in length (exclusive of the single-stranded overhang on the nonligating end), or for example from about fifteen to about fifty base pairs in length.

The cloning site of the oligomeric adapters can be any cloning site that enables joining of the adapter to another nucleic acid molecule, such as a DNA molecule, such as a restriction enzyme site, topoisomerase recognition site, or recombinase recognition site. The cloning site is preferably designed for high-efficiency cloning to maximize complexity of a library. For example, the integration site is preferably a topoisomerase recognition site or a recombination site that is recognized by a site-specific recombinase. Recombinational cloning, and recombination sites, enzymes, and formulations, methods for recombinational cloning, and in particular GATEWAY® cloning systems, reagents, and vectors (Invitrogen, Carlsbad, Calif.) are known in the art and disclosed in for example, U.S. Pat. Nos. 5,888,732; 6,171,861; 6,143,557; 6,270,969; 6,277,608; and U.S. Publication No. 2003 0124555, all of which are herein incorporated by reference for disclosure of cloning vectors, recombination sites, enzymes and reagents for cloning using recombination sites, and methods of cloning using recombination sites. For example, the adapters can be designed for cloning into vectors such as GATEWAY® vectors, and can have recombination sites such as, for example, an att site, such as an attB site, an attP site, an attL site, an attR site, an att1 site, an att2 site, or mutated recombination sites derived from any of these, such as but not limited to those described in PCT Application No. US 0005432 (published application WO 00/52027) herein incorporated by reference in its entirety. Other recombination sites that can be used include those used by transposases, resolvases, or the FLP/FRT system of S. cereviseae, as they are known in the art.

Methods of Generating Three Frame Expression cDNA Libraries

In another aspect, the present invention provides methods of cloning a three frame expression cDNA library using a three frame oligonucleotide adapter set of the present invention. The methods include joining separately joining each of three oligonucleotide adapters of a three frame oligonucleotide adapter set to three separate aliquots of a cDNA population to generate three distinct reading frame adapter-ligated cDNA library aliquots, in which each aliquot comprises a different reading frame adapter, and inserting the three distinct reading frame adapter-ligated aliquots of the cDNA library into an expression vector using a cloning site of the adapters. The expression vector can be a vector of any type and can be designed for replication in any cell type. The insertion of cDNA into an expression vector can be direct or indirect. For example, cDNA can be introduced into an expression vector by means of one or more intermediate vectors.

Methods of isolating RNA and for making cDNA from an RNA population are well known in the art. Methods of making cDNA libraries are also well known in the art, in which the methods may use any of a variety of reverse transcriptases and optionally other DNA polymerases, vectors for cloning the cDNAs, as well as adapters, linkers, restriction enzymes, and ligases or recombination enzymes for combining synthesized cDNA molecules with vectors. In some preferred embodiments of the invention, recombinational cloning is employed to insert cDNA molecules into expression vectors, and in these embodiments, the three frame adapters comprises recognition sites for recombination enzymes, or, simply, “recombination sites”. In some illustrative embodiments, GATEWAY® vectors and cloning reagents (including recombination enzymes) available from Invitrogen (Carlsbad, Calif.) are employed to generate expression cDNA libraries. GATEWAY® cloning methods and descriptions of vectors and adapters having recombination sites are provided, for example, in the manual “CLONEMINER™ cDNA Library Construction Kit” available at the Invitrogen.com web site.

The methods of the present invention in exemplary embodiments are in preferred embodiments directed toward making cDNA expression libraries for identifying proteins with activities of interest. To optimize expression of functional proteins, a primer for first strand synthesis that includes a poly (T) sequence and, 3′ of the poly (T) sequence, at least two nucleotides that are not part of the poly (T) sequence. The nucleotide immediately 3′ of the poly (T) sequence can be A, C, or G. The nucleotide that is 3′ of the poly (T) sequence and one base removed from the poly (T) sequence can be any of A, C, G, or T. Including one or, preferably two nucleotides 3′ of the poly (T) sequence can reduce the amount of poly T and 3′ UTR sequences in a cDNA by ensuring the primer hybridizes at the “upstream-most” end of an mRNA. The poly (T) sequence can be for example, more than 12, more than 15, more than 20, or about 25 T residues. The poly (T) sequence can be for example, less than 30 thymine (T) residues. In some preferred embodiments, a primer used for first strand synthesis has no more than 29, 28, 27, 26, or 25 T residues 5′ of the primer nucleotide that will hybridize to the 3′-most non-poly A residue of an mRNA. An example of a primer that can be used in the methods of the present invention for first strand synthesis in making an expression cDNA library is provided in Example 1.

Preferably the primer for cDNA synthesis includes sequences for cloning that are compatible with the cloning sequences in the 3-frame adapters, although this is not a requirement of the invention. Where adapters include restriction sites for cloning, for example, the primer can also include a restriction site, which can be the same or different from that of the adapter(s). In embodiments in which the adapters include recombination sites, the primer for cDNA synthesis, which becomes incorporated into the synthesized cDNA, preferably also includes recombination sites compatible with those of the adapters to facilitate cloning into an expression vector. In these preferred embodiments, the primer can optionally be biotinylated on its 5′ end. This is the end opposite the end that is extended in cDNA synthesis, and biotinylation can effectively cap or block the 5′ end of the primer (and synthesized cDNA) from being ligated to the adapters that are attached in a subsequent ligation step.

In another improvement to cDNA library synthesis methods, cDNA is size fractionated prior to cloning into an expression vector. In these methods, the synthesized cDNA is size fractionated to remove cDNAs larger than a particular size, such as, for example, larger than about 2.5 kilobases (kb) or larger than about 2 kb, or larger than about 1.5 kilobases kb, or larger than about 1 kb. This is contrary to common practice in which only cDNAs smaller than a certain size are excluded, and therefore referred to herein as “reverse size fractionated”. The inventors have found, however, that inclusion of larger cDNAs in expression libraries can result in cDNAs that include 5′-UTR regions that include stop codons being inserted into the expression vector, leading to a failure to express the cDNAs. In some embodiments, cDNAs larger than about 1.5 kb are excluded from the cloning pool. The upper size cutoff, above which cDNA is preferentially excluded from the population to be cloned, can be any thought to be useful or reasonable to the practitioner having knowledge of the experimental system and anticipated mRNA sizes, for example, about 0.5 kb, about 0.75 kb, about 1 kb, about 1.25 kb, about 1.5 kb, about 2 kb, about 2.5 kb, or about 3 kb or greater. Size fractionation preferably also includes a lower size cutoff, such that non-cDNA-ligated adapters and cDNAs less than a preferred length are preferentially excluded from integration into the cloning vector. The lower size cutoff can be, for example, about 0.1 kb, about 0.2 kb, about 0.5 kb, about 0.6 kb, about 0.7 kb, about 0.8 kb, about 0.9 kb, about 1.0 kb, or greater.

Size fractionation of cDNAs can be done by any means known in the art. For example, chromatography or gel electrophoresis, or a combination thereof, can be used for size fractionation. In some methods, a small amount of radiolabeled nucleotide is included in cDNA synthesis reactions to track cDNA loaded on a size fractionation column, which can include any sutiable matrix, as, for example, Sephacryl S-500 HR resin. Eluted cDNA fractions can be collected, and aliquots can be run on gels that include markers to determine which fractions have cDNA of the desired size range. Such methods are well known in the art for selecting large cDNAs, and are, for example, described in detail in the CLONEMINER™ cDNA construction kit instruction manual (available from Invitrogen.com). The methods can readily be adapted to selecting fractions that avoid cDNAs larger than a particular size.

In preferred aspects, cDNA three frame expression libraries are made by separately ligating a first adapter, a second adapter, and a third adapter of an adapter set of the present invention to aliquots of a population of cDNA molecules to generate a first, second, and third population of adapter-ligated cDNA molecules. The adapter-ligated cDNA molecules are integrated into a vector. The vector the adapter-ligated cDNA molecules are integrated into can be an expression vector, or can be a vector that is not designed for protein expression, and the cDNA library so generated can subsequently be transferred to an expression vector. For example, adapter-ligated cDNA molecules can be integrated into a vector that can be used to transform E. coli, which provides high transformation efficiencies, easy selection for cells containing vector, and convenient protocols for DNA isolation. The libraries generated in E. coli can optionally evaluated for complexity, presence of insert, and insert size in the E. coli vector. The three independently generated reading frame libraries generated in E. coli can optionally be pooled to form a pooled three-frame cDNA library. Alternatively, cDNAs ligated to different adapters of the three frame adapter set can be pooled prior to cloning into the expression vector, or recombined expression vector-3-frame adapter-ligated cDNAs of different adapter aliquots can be pooled prior to transformation of E. coli.

The integration of adapter-ligated cDNA molecules into a cloning vector is preferably performed by recombination, such as att site recombination that uses BP or LR reactions as used in the GATEWAY® cloning system (see for example, the GATEWAY® Technology manual, www.Invitrogen.com) that provides high efficiency cloning and the ability to easily transfer inserts from one GATEWAY® vector to another (such as, but not limited to, from an E. coli vector to a eukaryotic, for example, yeast or mammalian, vector. Recombinational cloning, and recombination sites, enzymes, and formulations, methods for recombinational cloning, and in particular GATEWAY® cloning systems, reagents, and vectors (Invitrogen, Carlsbad, Calif.) are known in the art and disclosed in for example, U.S. Pat. Nos. 5,888,732; 6,171,861; 6,143,557; 6,270,969; 6,277,608; U.S. Publication No. 2003 0124555, and PCT Application No. US 0005432 (published application WO 00/52027) all of which are herein incorporated by reference for disclosure of cloning vectors, recombination sites, enzymes and reagents for cloning using recombination sites, and methods of cloning using recombination sites.

The present invention also includes cDNA libraries made using the methods of the present invention, in which a population of cDNA molecules is synthesized using a set of three frame adapters that include a recombination site and cloned into a vector that contains recombination sites flanking the cDNA inserts.

The three-frame expression libraries generated in E coli can subsequently be transferred to a vector for expression in a cell type of interest, for example, a mammalian expression vector, a yeast expression vector, a plant cell expression vector, an insect cell expression vector, etc. In preferred aspects of the invention, three frame libraries are generated using adapters that include recombination sites, and transfer of a cDNA from one vector to another (for example a bacterial selection vector to a eukaryotic expression vector) can be performed using recombination sites on the cDNAs and vectors. For example, att recombination sites integrated into adapters and GATEWAY® vectors that are designed for efficiently moving DNA fragments from vector to vector are used in preferred embodiments of the invention. In these embodiments, recombination sites that are incorporated into the three frame adapters provide for the initial insertion of cDNA molecules into a vector, such as a bacterial vector, and the (subsequently altered) recombination sites are further used for transferring the cDNA library sequences into expression vectors for use in other systems.

In some aspects of the present invention, the expression vectors are designed for in vitro expression systems. In vitro transcription/translation systems can use prokaryotic or eukaryotic cell extracts. In other aspects of the invention, the expression vectors are designed for expression within cells, such as but not limited to, bacterial cells, yeast cells, Xenopus cells, zebrafish cells, insect cells, plant cells, or mammalian cells.

The vector can be configured such that cDNAs of the library are expressed as tagged proteins or fusion proteins. For example, the expression vector can include sequences that encode a tag such as a his tag, a myc tag, a FLAG tag, etc., glutathione S-transferase or a domain thereof streptavidin or a domain thereof a chitin binding protein, calmodulin or a domain thereof, maltose binding protein or a domain thereof, etc. The vector is configured such that the tag is linked to the protein expressed by cDNA cloned in the vector. The vector can also include reporter proteins or selectable marker proteins, such as but not limited to a fluorescent protein, a lumio sequence, an enzyme such as beta galactosidase or GUS, or a protein that confers resistance or sensitivity to a drug or compound, or is an auxotrophic marker. The protein encoded by cDNA cloned in the vector can be synthesized as a fusion protein with a reporter protein or selectable marker. Fusion proteins or protein domains or tags can be designed to be linked to either the N-terminus or C-terminus of a protein encoded by a cDNA of the library. In exemplary embodiments, fusion proteins, domains, or tags are provided in an expression vector having a cloning site compatible with that of the adapters, where the cDNA of the library is cloned C-terminal to the fusion protein, domain, or tag.

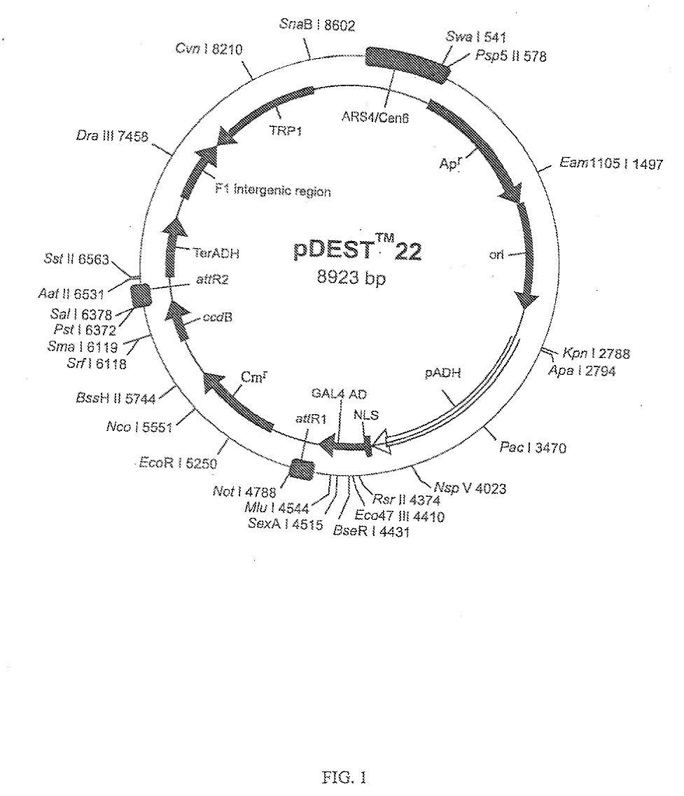

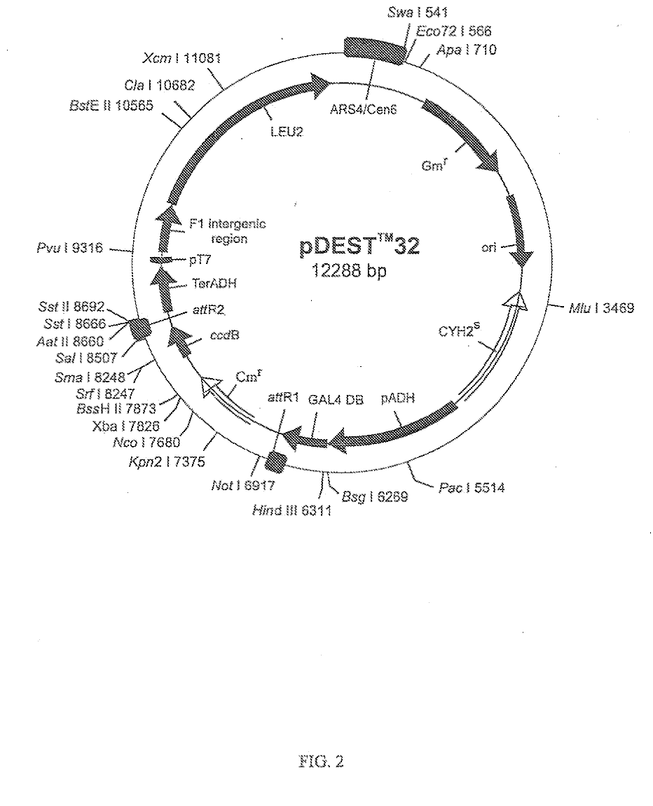

In some preferred embodiments, an expression vector used in the methods of the present invention encodes a functional domain of a protein, such that expression of clones of the library directs the synthesis of fusion proteins that include the protein encoded by the cDNA fused to an active domain of a known protein. In preferred embodiments in which the libraries synthesized using the methods of the present invention are used for identifying for proteins that interact with a protein of interest in two hybrid screens, the cDNA library is cloned in an expression vector that includes sequences that encode either a DNA binding domain (DBD) or a transcriptional activation domain (AD). In an illustrative embodiment, the cDNA library is introduced in to pDEST™ 22, depicted in FIG. 1, and a construct is made in which a “Bait” protein is fused to the domain of expression plasmid pDEST 32™ (shown in FIG. 2).

In some preferred embodiments, the libraries are provided in expression vectors that include sequences that direct transcription and translation of a DNA sequence cloned in the vector are well known in the art. The expression vector can be any expression vector designed for expression in any type of cells, prokaryotic or eukaryotic. For example, the vector can be a bacterial, yeast, mammalian, insect, or plant expression vector. The vector can be designed for expression in in vitro systems, such as in vitro protein synthesis system, including prokaryotic or eukaryotic in vitro transcription and translation systems. A preferred vector is a GATEWAY® expression vector.

The present invention provides three frame cDNA expression libraries in expression vectors that include additional protein coding sequences that are fused to cDNA sequences. For example, three frame expression cDNA libraries of the present invention can be in GATEWAY® expression vectors, such as but not limited to pDEST 22 (FIG. 1) or pDEST 32 (FIG. 2), that include sequences encoding the GAL4 DBD or AD, respectively, for use in two hybrid screens in yeast cells. cDNA libraries can be cloned into entry vectors (such as, but not limited to, the pEXP22 vector replicates in both yeast and E. coli and that has an AD for two hybrid screens) and subsequently transferred in recombination reactions, to other expression vectors (such as but not limited to pDEST 22) for functional screening.

The libraries can be made from RNA from any source, from prokaryotic cells or eukaryotic cells. In some embodiments, tissue-specific mammalian libraries are provided, such as human spleen, kidney, heart, or skeletal muscle three frame expression cDNA libraries provided in a pDEST 22 vector.

III. Screens for Proteins Having an Activity of Interest

Another aspect of the present invention is a method of performing a screen for protein activity using a cDNA three frame expression library of the present invention. The screen can be in vitro or in vivo and can be a screen for a protein activity or a cellular phenotype or activity. For example, an in vitro screen can be a binding assay, or an enzyme assay, such as but not limited to a kinase assay. In vivo screens can be any type of in vivo assay, such as but not limited to: a binding assay, a gene expression assay, an ion channel or ion transporter assay, a GPCR assay, a cell growth assay, an apoptosis assay, or a cell migration assay.

For example, binding assays or enzymatic assays can be performed using proteins generated using in vitro or in vivo translation of expression libraries of the invention (see for example, U.S. Pat. Nos. 5,654,150 and 6,274,321, herein incorporated by reference in their entireties for all disclosure of methods of in vitro screening of synthesized proteins).

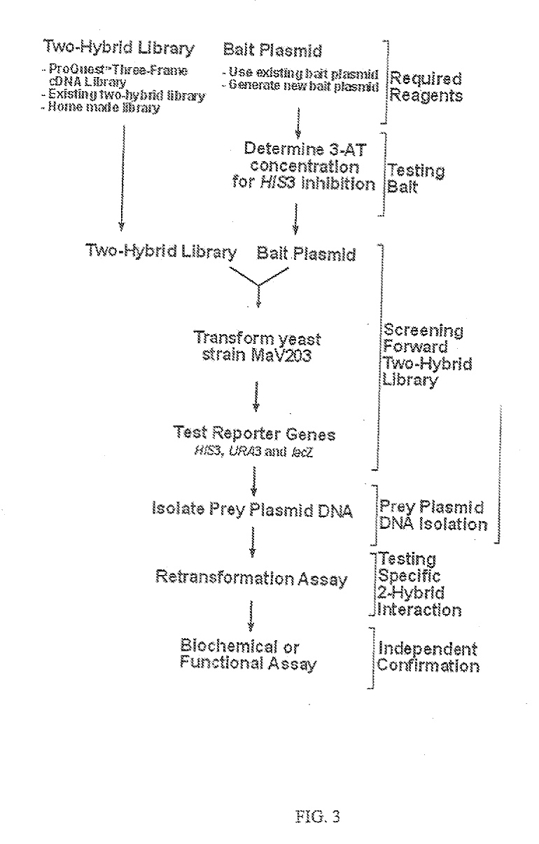

The compositions and methods of the present invention find particular utility in two hybrid assays that screen for proteins that interact with a protein of interest. Two hybrid screens can be performed in any type of cell. Preferred cells for two hybrid screens are yeast cells and mammalian cells. Two hybrid screens are well-known in the art (see for example, U.S. Pat. Nos. 5, 283,173, 5,468,614, and 5,667,973, all herein incorporated by reference for all disclosure of yeast two hybrid systems, host strains, reagents, and methods). Methods of two hybrid screens in yeast and description of GATEWAY VECTORS that can be used in the screens can also be found, for example, in the manual “PROQUEST™ Two Hybrid System” available at Invitrogen.com. FIG. 3 is a flow chart of steps in two-hybrid screening.

For example, three frame cDNA libraries made using the methods of the present invention can be cloned (for example, using GATEWAY® technology) into an expression vector (such as, but not limited to, pDEST 22), downstream of sequences encoding an AD (such as the GAL4 AD), such that a cDNA ORF is fused to the AD. A “prey” protein of interest can be provided in the yeast strain in an expression vector (such as, but not limited to, pDEST 32), downstream of and fused to a DBD (such as the GAL4 DBD). (The assay can also be performed with the protein of interest fused to an AD and library ORFs fused to a DBD.) The yeast cells expressing the protein of interest fused to a DBD are transformed with the three frame cDNA expression library. Proteins encoded by cDNAs of the library are assayed for their interaction with the protein of interest by detecting the expression of one or more selectable markers or reporter genes by the yeast cells transformed by the three frame expression library.

The methods of the present invention that optimize the number of expressed sequences of an expression library can be used in any methods that survey expression libraries for proteins having properties of interest.

For example, another type of screen that can be performed using the three frame expression libraries of the present invention is a “three hybrid” screen to find proteins that interact with a compound of interest. For example, a compound that has effects on cells but whose target is not known can be directly or indirectly conjugated or bound to a protein that includes or is bound to a DBD or AD. Three frame expression cDNA libraries, in which the cDNAs are expressed as fusion proteins that also include an AD or DBD (whichever is not bound to the compound of interest) can be transformed into cells that contain the compound conjugate to screen for proteins that interact with the compound, thereby activating expression of a reporter or marker gene, where the reporter or marker gene is activated by the DBD bound by the AD. cDNAs that activate reporter or marker gene expression when transformed into the cells putatively encode proteins that interact with the compound, and are candidate compound targets. Methods of performing two hybrid screens for proteins that interact with a compound are described, for example, in WO 02070662, U.S. Publication No. 20040043388, U.S. Publication No. 20030165873, WO03033499, and U.S. Publication No. 20050090471, all herein incorporated by reference for all disclosure of compositions, systems, and methods for performing three hybrid screens.

Kits

The invention includes kits that include 3 reading frame adapter sets as described herein. The adapters can be provided together in separated containers as liquid solutions or solids (e.g., lyophilates or precipitates). The 3 adapters of a 3 reading frame adapter set can be packaged together in separated containers.

Kits can also include one or more primers for cDNA synthesis. A primer provided in a kit preferably includes a poly (T) sequence, and is preferably a primer as described herein that includes a cloning site, and can have, for example, less than 30 T's followed by (3′ of the T's) one or more, such as 2, residues that are not part of the poly T sequence at the 3′ terminus of the primer.

Kits can further include a vector, such as but not limited to a GATEWAY® vector. The GATEWAY® vector provided in a kit can be an entry vector, or can be an expression vector, and can be an expression vector that includes an AD or a DBD to which a cloned cDNA can be fused, such as, for example, pDEST 22 or pDEST 32.

Kits can optionally further include one or more reagents for cDNA synthesis, cloning, or screening, and/or one or more reagents, such as but not limited to selectable markers, for two hybrid screening.

The following examples are intended to illustrate but not limit the invention.

### EXAMPLE 1

### cDNA Expression Library Synthesis

Three-frame Libraries: The following describes the construction and qualification of 2-hybrid entry libraries that are expected to be enriched for in-frame ORFs via the introduction of adapters containing 3 possible reading frames. The CLONEMINER™ cDNA synthesis kit (Invitrogen) has been modified for these libraries to include a new oligo d(T) primer designed to reduce the length of 3′ polyadenylation sequences and the incorporation of three separate cDNA-adapter ligations to allow for the possibility of 3 reading frames within the completed library. In addition, the sizing of cDNA prior to BxP recombination is performed as to reduce 5′ UTR regions which may contain stop codons and reflect a smaller average insert size (AIS) than the standard CLONEMINER™ library construction method (Invitrogen, Carlsbad, Calif.). All libraries are constructed in pDONR 222 (Invitrogen) which allows for subsequent LxR transfer into destination vectors.

RNA Samples Used for cDNA Library Construction

All RNA samples were purchased from BioChain Institute Inc. (Hayward, Calif.; Biochain.com) Information is listed in Table 1.

TABLE 1
RNA Populations Used to Make Three-Frame cDNA Libraries

| | | | | | Donor | Donor |
|---|---|---|---|---|---|---|
| Cat # | Lot# | Species | Type | Tissue | Sex | Age (y) |
| R1234035 | A607141 | Human | Normal Adult | Brain | M | 28 |
| R1255811-50 | A611271 | Cell Line | | Hela | | |
| R1244035 | A611053 | Human | Fetal | Brain | F | 22 wks |
| R1234149 | A608265 | Human | Normal Adult | Liver | M | 64 |
| R1234086 | A611274 | Human | Normal Adult | Breast | F | 78 |
| R1234148-50 | A607343 | Human | Normal Adult | Peripheral Blood Leukocyte | M | 38 |
| R1234148-50 | A607341 | Human | Normal Adult | Peripheral Blood Leukocyte | M | 37 |
| R1234122 | A608164 | Human | Normal Adult | Heart | M | 24 |
| R1234142 | A607166 | Human | Normal Adult | Kidney | M | 46 |
| R1234090 | A611275 | Human | Normal Adult | Colon | M | 24 |
| R1234152 | A607137 | Human | Normal Adult | Lung | M | 24 |
| R1234246 | A609288 | Human | Normal Adult | Spleen | M | 30 |
| R1234201 | A610090 | Human | Normal Adult | Prostate | M | 34 |
| R1234183 | A610304 | Human | Normal Adult | Ovary | F | 51 |
| R1234171 | A607190 | Human | Normal Adult | Skeletal Muscle | M | 44 |
| R1234188 | A608226 | Human | Normal Adult | Pancreas | M | 62 |
| | 8906030 | Human | Hyperplasia | Prostate (mRNA) | | |
| | 8906028 | Human | Normal Adult | Placenta (mRNA) | | |

Three Reading Frame Entry cDNA Library Construction

The cDNA library construction was performed using a Standard BxP Library Protocol (a modified CloneMiner System, Invitrogen) with three modifications as following:

Primer for the 1^{st} Strand cDNA Synthesis:

The Biotin-attB2-d(T)₂₅ VN primer in 2 hybrid 3 reading frame entry library is substituted for Biotin-attB2-d(T)₁₉ primer. The sequence of oligo d(T) VN₂₅ primer is:

(SEQ ID NO:1)

5′-Biotin-ACAACTTTGTACAAGAAAGTTGGGTGCGGCCGC
(T)₂₅VN-3′
Where V = C, G, A; N = C, G, A, T

Adapters:

Three adapters each containing the attB1 site are used in the construction of each 2-hybrid library. The sequences of the 3 reading frame adapter oligomers are as follows: (Note: Reading frame alpha adapter is the adapter from the CloneMiner kit, Invitrogen, Carlsbad, Calif.).

RF alpha:

(SEQ ID NO:2)

5′-TCGTCGGGGACAACTTTGTACAAAAAAGTTGG-3′
(long strand)

(SEQ ID NO:3)

3′-CCCCTGTTGAAACAGTTTTTTCAACCp-5′
(short strand)
RF beta:

(SEQ ID NO:4)

5′-TCGTCGGGGACAACTTTGTACAAAAAAGTTGGA-3′
(long strand)

(SEQ ID NO:5)

3′-CCCCTGTTGAAACAGTTTTTTCAACCTp-5′
(short strand)
RF gamma:

(SEQ ID NO:6)

5′TCGTCGGGGACAACTTTGTACAAAAAAGTTGGAA-3′
(long strand)

(SEQ ID NO:7)

3′-CCCCTGTTGAAACAGTTTTTTCAACCTTp-5′
(short strand)

The ‘sense’ long strand oligo of the adapters is 5 bp longer than the short strand oligo. The short oligomer is phosphorylated at 5′end.

1^{st} Strand Synthesis:

Solution A:

| mRNA (2 ug) | ( ) | ul |
|---|---|---|
| 10 pmol/ul Biotin-attB2-dT(25)VN | 1 | ul |
| 10 mM dNTP | 1 | ul |
| DEPC H₂O | ( ) | ul |
| Total | 11 | ul |

Heat to 65° C. for 5 min and cool to 45° C. in PCR cycler or water bath.

Solution B

| | |
|---|---|
| 5X 1^{st} strand buffer | 4 ul |
| 0.1 M DTT | 2 ul |
| DEPC H₂O | 1 ul |
| Total | 7 ul |

Mix Solution A and B at 45° C. in PCR cycler or water bath, theniIncubate at 45° C. for 2 min. Add 2 ul Superscript III RT (Invitrogen) (200 u/ul) and mix. Incubate at 45° C. for 60 min, cool to 16° C., and then remove 1 ul from 1^{st} strand reaction and mix with 24 ul of 20 mM EDTA for calculation of 1^{st} strand incorporation.

2^{nd} strand synthesis

| | | |
|---|---|---|
| 1^{st} strand cDNA | 19 | ul |
| DEPC H₂O | 92 | ul |
| 5X 2^{nd} strand buffer | 30 | u |
| 10 mM dNTPs | 3 | ul |
| E. coli ligase | 1 | ul |
| E. coli DNA polymerase I | 4 | ul |
| E. coli RNase H | 1 | ul |
| Total | 150 | ul |

Mix well (gently) and incubate at 16° C. for 2 hours. Perform the calculation of 1^{st} strand cDNA incorporation as shown below. Add 1 ul T4 DNA polymerase. Incubate at 16° C. for 5 min, then add 10 ul of 0.5 M EDTA. Add 160 ul phenol/chloroform and extract after extraction, add 1 ul glycogen, 80 ul 7.5 M ammonium acetate, 600 ul ethanol, place on dry ice for 30 min and spin. Wash twice with 70% ethanol. Completely resuspend (20 times) in 81 ul 1×TE.

attB1 Adapter Ligation:

Set up three individual reactions in separated tubes: alpha, beta, and gamma. Each with 27 ul of attB2-cDNA, 10 ul of 5× Adapter buffer, 1 ul of 1 ug/ml att B1 adapter (alpha, beta, and gamma, respectively), 7 ul of 0.1 M DTT, and 5 ul of T4 DNA ligase (1 unit per ul). Incubate at 16° C. overnight (16-24 hours). Heat to 70° C. for 10 min. and cool on ice. Add 100 ul of TEN buffer and keep on ice.

Column Fractionation:

A separate column is used for each library aliquot (alpha, beta, and gamma adapter-ligated). Wash DNA purification column with 0.8 ml of TEN 4 times. Load 150 ul DNA to the column. Drain to Tube 1. Load 100 ul TEN. Drain to Tube 2. Load 100 ul TEN and collect one drop (about 40 ul) in each tube into Tubes 3-10. Count cpm of aliquots of each fraction, calculate amounts and volume in each tube. Combine Tubes 5 to 8 (about 160 ul). Add 1 ul of 20 mg/ml glycogen to each tube, 0.5 volume of 7.5 M ammonium acetate, 2.5 volume of ethanol. Freeze on dry ice for 30 min or −20 c for O/N, spin, and wash twice with 70% ethanol. Dry pellets in the Spin Vacuum for 2 min. Resuspend in 4 ul T10E0.1.

BXP Reaction:

| | |
|---|---|
| DNA sample | 4 ul |
| pDONR222 (250 ng/ul) | 1 ul |
| 5X BXP Buffer | 2 ul |
| BXP CLONASE ™ enzyme mix | 3 ul |
| Total | 10 ul |

Incubate at 25° C. for O/N (16-24 hours). Don't let reaction solution condense on cap. Add 1 ul of proteinase K. Incubate at 37° C. for 15 min, and then 75° C. for 10 min. Add 90 ul of H₂O, 1 ul of glycogen (20 ug/ul), 50 ul of 7.5 M ammonium acetate, 375 ul of ethanol. Freeze on dry ice, spin, and wash twice with 70% ethanol. Dry pellets. Resuspend in 9 ul of T10E0.1.

Electroporation:

Add 1.5 ul of cDNA to 80 ul of DH10B, mix and transfer into cuvette. Electroporate at 2.5 kv, 100 Ohm, 25 uF. Add 920 ul of SOC, shake at 37° C. for 1 h. Add equal volume solution of 40% glycerol/60% SOC. Remove 100 ul and transfer into a fresh 1.5 ml tube and make serial dilutions as 1×10⁻¹, 1×10⁻², 1×10⁻³ in 1.5 ml of tubes. Plate 100 ul on each plate. After overnight incubation of plates, count the colonies on each plate to determine the library titer and total cfu of the library.

Quality Testing of Three Reading Frame Entry Libraries

% Insert and Average Insert Size (AIS): AIS can be determined by: 1) Colony PCR on 24 individual colonies using M13 forward or entrF1 primers and M13 reverse primer to determine the number of inserts and establish the average insert size (AIS); and 2) BsrGI digestion on 24 DNA samples derived from 24 colonies if PCR is not successful.

Colony PCR

Pick individual colonies by using a small pipet tip to touch the middle of each colonies, mix the tip 3-4 times in each well of 96 well plate (Master Plate) that contain 25 ul clean water per well. Dispense all solution into Master Plate and transfer the tips into another 96 well plate (PCR Plate) and mix with PCR reagents prepared as following: 5 ul 10× pCR buffer, 1 ul M13 F or entrF1 primer (20 pmol/ul), 1 ul M13R primer (20 pmol/ul), 0.5 ul 10 mM dNTP mix, 1 ul PCR enzyme (Taq), and 16.5 ul sterile water. Perform PCR using the following conditions: 1 cycle at 94° C. for 2 minutes; 2) 30 cycles at 94° C. for 30 seconds, 45° C. for 30 seconds and 72° C. for 3 minutes; 3) 1 cycle at 72° C. for 10 minutes. Add 2.5 ul 10× loading dye and run 10 ul per lane on a 0.8% agarose gel against 1 kb plus marker.

BsrGI Digestion

Isolate plasmid DNA using SNAP or QIAGEN 96 well miniprep following manufacture protocols. Digest 1-2 ug of DNA for each sample with BsrG1: 15 ul DNA, 3 ul NEB #2 buffer, 0.3 ul BSA, 1 ul BsrG1 enzume, and 5.7 ul water. Incubate at 37° C. for 1 hour. Add 2.5 ul 10× loading dye and run 10 ul on 0.8% agarose gel against 1 kb plus MW ladders.

Preparing Entry Library DNA

Once the titers of individual reading frame entry libraries have been determined and they have passed the QC for insert size and AIS, an equal amount of cfu of each reading frame library is inoculated into a 100 ml culture of LB supplemented with Kan₅₀. Grow overnight at 30° C. with shaking at 225 rpm. Preferably at least 2×10⁶ cfu is used for each entry library culture.

DNA Preparation

A midiprep of each culture is prepared. A SNAP or QIAGEN kit may be used for this purpose. However, if SNAP or other kits are used, the eluted DNA should be extracted once with phenol:chloroform:isoamyl alcohol (25:24:1) to further clean DNA samples and resuspended in ˜100 ul TE.

DNA Quality

DNA amount can be determined by OD_{260/280} of each DNA prep. The _{260/280} ratio should be at least 1.7. Cut 500 ng of entry library with BsrGI and run the BsrGI digested sample and 500 ng of uncut DNA sample on a 0.8% agarose gel to verify quality.

Final Entry Library DNA Sample

Combine an equal amount of DNA from each reading frame library into a separate tube. This is the final library DNA to be used for subsequent LxR transfers into yeast and mammalian 2-Hybrid vectors.

Sequencing Analysis to Verify the Ratio of Three Reading Frames

Sequencing may be performed on colonies from the mixed 3rf Entry library or DEST library to verify the presence of 3 reading frames in the library and their respective ratios. 5′ end sequencing may be done with M13 forward or EntrF1 primers.

Results of cDNA Library QC

Primary Entry cDNA Library in pENTR222

PCR or BsrGI digests were performed on 24 clones from each reading frames (alpha, beta, gamma) of entry library. The titer, % inserts, AIS (average insert size) and total cfu (colony forming units) numbers used for DNA preparation from one example of Hela library are shown in Table 2.

TABLE 2
Hela Entry Three Reading Frame Library

| | | | | Total cfu |
|---|---|---|---|---|
| Entry library | Titer | % inserts | AIS | in DNA prep |
| Alpha frame | 5.5 × 10e5 cfu/ml | 100 | 1.6 kb | 3.0 × 10e6 |
| Beta frame | 1.8 × 10e6 cfu/ml | 100 | 1.4 kb | 3.0 × 10e6 |
| Gamma frame | 1.9 × 10e5 cfu/ml | 100 | 1.6 kb | 3.0 × 10e6 |

QC by colony PCR to determine the % inserts and AIS from 24 individual clones from a mixed three reading frames entry library was performed. 24 out of 24 clones contain inserts (100%) and AIS is 1.4 kb.

QC of First Strand cDNA System.

The QC of first strand cDNA synthesis can be monitored by loading a small portion (2 ul) of 1^{st} strand cDNA on a agarose gel to check the quality and yield, for example on an 0.8% agarose gel with 0.1 % ethidium bromide.

QC of Entry Library DNA

Digest 0.5-1 ug of prepared DNA from entry library with BsrGI as follows: 5 ul DNA, 3 ul NEB #2 buffer, 0.3 ul BSA, 1 ul BsrG1 enzume, and 5.7 ul water. Incubate at 37° C. for 1 hour. Add 2.5 ul 10× loading dye and run 10 ul on 0.8% agarose gel against 1 kb plus MW ladders. A band at 2.5 kb that is the backbone of pENTR222 vector should be visible as well as a smear ranging from 0.1 to 10 kb that contains cDNA inserts of different sizes.

Synthesized Three Frame cDNA Expression Libraries for Two Hybrid Screens

Using the methods described above, three frame expression cDNA libraries were generated in pDEST22. The libraries met the following criteria: library titers were ≦5×10⁹ cfu/ml; the average insert size by BsrG1 digestion of 24 clones was between 0.5-1.7 kb; and at least 21 out of 24 tested clones contained inserts.

### EXAMPLE 2

Kit for Three Frame cDNA Expression Libraries Synthesis

PROQUEST™ Three-Frame cDNA Libraries have been constructed using the CLONEMINER™ cDNA Library Construction Kit, which eliminates use of restriction enzyme digestion and ligation allowing cloning of undigested cDN and allows highly efficient recombinational cloning of cDNA into a donor vector results in a higher number of primary clones compared to standard cDNA library construction methods (Ohara & Temple, 2001), while enabling highly efficient transfer of a cDNA library into multiple destination vectors for protein expression and functional analysis.

PROQUEST™ Three-Frame cDNA Libraries are constructed using three frame 5′ adapters instead of the single adapter provided in the CLONEMINER™ cDNA Library Construction Kit. These adapters differ by one and two nucleotides in length to permit expression of ORFs in all the 3 possible reading frames.

The sequences of the 3 reading frame adapter oligos containing the attB1 recombinational cloning site are as follows:

Reading Frame alpha:

(SEQ ID NO:2)

5′-TCGTCGGGGACAACTTTGTACAAAAAAGTTGG-3′

(SEQ ID NO:3)

3′-CCCCTGTTGAAACAGTTTTTTCAACCp-5′
Reading Frame beta:

(SEQ ID NO:4)

5′-TCGTCGGGGACAACTTTGTACAAAAAAGTTGGA-3′

(SEQ ID NO:5)

3′-CCCCTGTTGAAACAGTTTTTTCAACCTp-5′
Reading Frame gamma:

(SEQ ID NO:6)

5′TCGTCGGGGACAACTTTGTACAAAAAAGTTGGAA-3′

(SEQ ID NO:7)

3′-CCCCTGTTGAAACAGTTTTTTCAACCTTp-5′

Reduced 5′UTRs

The CLONEMINER™ cDNA Synthesis Kit contains a size fractionation step that generates cDNA free of adapters and other low molecular weight DNA. In the construction of the PROQUEST™ Three-Frame cDNA Libraries, the largest cDNAs are also excluded, which serves to reduce 5′ UTR regions that may contain stop codons. This is reflected in a smaller average insert size (1-1.5 kb) than the standard CLONEMINER™ library construction method.

Reduced Poly-A Sequences

The CLONEMINER™ cDNA Synthesis Kit has been modified to reduce the length of 3′ polyadenylation sequences. Two nucleotides (VN) have been added to the 3′ end of the oligo d(T) primer to anchor the 1^{st} strand cDNA synthesis to the start of the Poly-A tail. The sequence of oligo d(T)₂₅VN primer is:

(SEQ ID NO:1)

5′-Biotin-ACAACTTTGTACAAGAAAGTTGGGTGCGGCCGC
(T)₂₅VN-3′
Where V = C, G, A; N = C, G, A, T

Preparation of PROQUEST™ Libraries

PROQUEST™ Three-Frame cDNA Libraries are prepared as follows: 1) mRNA is isolated using two steps. First, total RNA is isolated from tissues using the TRIzol® Reagent. Second, mRNA is isolated from total RNA using the FastTrack™ MAG mRNA Isolation Kit (Invitrogen, Carlsbad, Calif.).

2) cDNA is synthesized using a modified CLONEMINER™ cDNA Library Construction System: First-strand cDNA is synthesized using Biotin-attB2-Oligo(dT)-VN primer, followed by second-strand cDNA isynthesized using E. coli RNase H, E. coli DNA polymerase I and E. coli DNA ligase. Blunt-end cDNA is created using T4 DNA polymerase, and the cDNA is divided into three portions to be adapted with three different reading frame attB1 adapters (alpha, beta and gamma). Adapters beta and gamma contain one and two more base pairs respectively at the C-terminal end of the adapters. Three frame cDNAs are separately size-selected using column chromatography, and then size-selected cDNAs are separately cloned into the pDONR™222 vector through a GATEWAY® BP recombination reaction.

The BP recombination mix is transformed into ELECTROMAX™ DH10B™-T1^{R} E. coli and the number of primary recombinants is determined. An equal amount of library DNA from recombinants generated in each reading frame is mixed and transferred into pDEST™22 vector by GATEWAY® LR recombination. The LR recombination reaction is transformed into ELECTROMAX™ DH10B™-T1^{R} competent E. coli and number of primary recombinants is determined. The cDNA library is amplified once using a semi-solid procedure (Kriegler, 1990) to minimize representational biases.

pEXP22 is an activation domain expression vector which contains the GATEWAY® recombination sites attB 1 and attB2. The vector is generated by recombinational cloning of an insert into pDEST22. The major features of the vector are: a constitutive, moderate-strength yeast alcohol dehydrogenase (ADH1) promoter for expression of GAL4 fusions, the SV40 large T antigen nuclear localization sequence, the GAL4 activation domain (AD) allowing expression of the reporter gene which is activated when brought into proximity with the DNA binding domain by interacting bait and prey proteins, the recombination sites, attB1 and attB2 for transfer of cDNA into GATEWAY® recombinational cloning-compatible vectors, the ADH1 transcription termination (TT) for efficient transcription termination and stabilization of the mRNA, an f1 origin for ss DNA production, the TRP1 gene for auxotrophic selection of the plasmid in Trp⁻ yeast hosts, an ARSH4/CEN6 sequence for replication and low-copy number maintenance of plasmid in yeast, an ampicillin resistance gene for selection of transformants in E. coli, and the pUC origin for high copy replication and maintenance of the plasmid in E. coil

Preparing dsDNA from cDNA Library

Materials: Terrific Broth and PURELINK™ HiPure Plamid Midiprep Kit (Invitrogen, Carlsbad, Calif.) or other DNA preparation kit or materials.

Inoculate 100 ml Terrific Broth containing 100 μg/ml ampicillin with 2.5×10⁹ cells from the library in a 500-ml flask. Incubate the culture for 16 hours at 30° C. with shaking at 275 rpm. Read the A₅₉₀ of the culture. For accurate A₅₉₀ determination, dilute the cells 1:10-1:20, so that the observed value is between 0.2 and 0.8. In two 50-ml centrifuge tubes, process ˜500 OD₅₉₀ units. Centrifuge the tubes at 4800×g for 15 minutes at 4° C. Discard the supernatant. Prepare DNA according to the instructions provided with the PURELINK™ HiPure Plamid Midiprep Kit (Invitrogen, Carlsbad, Calif.), or other DNA preparation kit or materials.

Colony PCR Screening

A colony PCR procedure to screen for the presence of specific cDNA is described below. This method can also be used to identify desired cDNA clones, or amplify out the insert. cDNA specific primers can be used to screen for a specific cDNA, or identify desired cDNA clones. If you want to amplify out the insert, the sequences of the suggested PCR primers are shown below.

Suggested forward sequencing/PCR primer

(SEQ ID NO:8)

5′-TATAACGCGTTTGGAATCACT-3′

Suggested reverse sequencing/PCR primer

(SEQ ID NO:9)

5′-AGCCGACAACCTTGATTGGAGAC-3′

Add 10 μl TE to each labeled, 0.5 ml microcentrifuge tube. Pick individual colonies using a pipette tip and place the colonies directly into separate tubes containing TE. Pipette up and down to mix. Incubate the tubes in a pre-warmed thermal cycler at 99° C. for 5 minutes. Incubate the tubes on ice for 2 minutes. Centrifuge briefly to collect the sample at the bottom of the tube. Replace the tubes on ice. Prepare the appropriate amount of the following reaction mix and add 40 μl of reaction mix to each tube: 1× PCR Buffer (contains no MgCl₂), 0.2 mM dNTP mix, 0.5 μM primers, 2.4 mM MgCl₂, 2.5 units Platinum® Taq DNA polymerase. Bring the volume to 50 μl with sterile water.

Perform PCR using the following cycling parameters: 95 degrees C. for 2 minutes; followed by 40 cycles of: 1 minute at 94 degrees C., 1 minute at 55 degrees C., and 1 minute at 72 degrees C.; followed by a final incubation of 5 minutes at 72 degrees C.

Transfer 10 μl of each reaction to a new tube containing 2 μl 10× gel loading buffer (such as 10× BLUEJUICE™ Gel Loading Buffer (Invitrogen, Carlsbad, Calif.) Electrophorese the samples on a 1.5% agarose gel and analyze results.

PROQUEST™ Two-Hybrid System

The PROQUEST™ Two-Hybrid System is an in vivo yeast-based system for identifying protein-protein interactions (Chevray & Nathans, 1992). The major features of the system are: Low copy (ARS/CEN) vectors for reduced toxicity, three reporter genes with independent promoters to reduce false positives due to non-specific interactions, GATEWAY® recombinational cloning-compatible vectors for transferring your DNA sequences of interest into a variety of expression and analysis vectors. More details on screening PROQUEST™ Three-Frame cDNA Libraries using the PROQUEST™ Two-Hybrid System, can be found in the PROQUEST™ Two-Hybrid System manual available for downloading from the Invitrogen.com web site.

GATEWAY® Recombinational Cloning

The vector, pEXP22 contains attB1 and attB2 recombination sites flanking the cDNA cloning site. The cDNA insert can be transferred into other GATEWAY®-compatible vectors for expression by performing a BP recombination reaction with a pDONR™ vector. Further details on the GATEWAY® Technology, are available in the GATEWAY® Technology with CLONASE™ II Manual on the Invitrogen.com web site.

All references cited herein, including world wide web sites and documents, literature references, patent applications, and patents, are incorporated by reference herein in their entireties for all purposes.

Features of embodiments disclosed herein can be combined to make further embodiments that are also within the scope of the invention. Headings are for the convenience of the reader only, and do not limit embodiments of the invention. Although the invention has been described with reference to the above examples, it will be understood that modifications and variations are encompassed within the spirit and scope of the invention. Accordingly, the invention is limited only by the following claims.

We claim:

1. A method of making a three frame cDNA expression library, comprising:
ligating a first adapter of a three frame adapter set to a first aliquot of a population of cDNA molecules, ligating a second adapter of a three frame adapter set to a second aliquot of the population of cDNA molecules, and ligating a third adapter of a three frame adapter set to a third aliquot of the population of cDNA molecules, to generate least three aliquots of the population of cDNA molecules; and
inserting the three aliquots of the population of cDNA molecules into an expression vector to generate a three frame cDNA expression library.

2. The method of claim 1, further comprising transforming the cDNA expression library into a host cell.

3. The method of claim 1, wherein the cells are bacteria, yeast, mammalian, or insect cells.

4. The method of claim 1, further comprising, prior to ligating, reverse transcribing a population of RNA molecules with a primer that includes a poly (T) sequence.

5. The method of claim 4, wherein the primer further includes, 3′ of the poly (T) sequence, at least two non-poly T nucleotides.

6. The method of claim 1, further comprising reverse size-selecting the population of cDNA molecules prior to inserting the population of cDNA molecules into the vector.

7. The method of claim 4, wherein the reverse transcribing uses an primer that comprises more than 20 Ts.

8. The method of claim 1, wherein the reverse transcribing uses an oligo dT primer that comprises 25 Ts.

9. The method of claim 1, wherein the adapters comprise cloning sites.

10. The method of claim 9, wherein the adapters comprise restriction sites, topoisomerase recognition sites, or recombination sites.

11. The method of claim 10, herein the primers comprise recombination sites.

12. The method of claim 11, wherein the recombination sequences are att sites.

13. The method of claim 7, wherein the primer comprises a cloning site.

14. The method of claim 14, wherein the primer comprises a recombination site.

15. The method of claim 14, wherein the primer comprises an attB site.

16. A method of making a three frame expression cDNA library, comprising:
synthesizing a population of cDNA molecules from a population of RNA molecules;
dividing the population of cDNA molecules into at least three cDNA aliquots;
ligating a first adapter oligomer to a first cDNA aliquot,
ligating a second adapter oligomer to a second cDNA aliquot, and ligating a third adapter oligomer to a third cDNA aliquot,
inserting each of the cDNA aliquots into a vector to generate at least one vector cDNA library; and
transforming the three vector cDNA libraries into bacteria to obtain at least one cDNA expression library.

17. A set of three frame adapters for making a cDNA expression library, comprising:
A first adapter having ‘n’ nucleotides;
A second adapter having ‘n+(3)i+1’ nucleotides, and
A third adapter having ‘n+(3)i+2’ nucleotides.

18. The set of three frame adapters of claim 17, comprising wherein each of the adapters comprises a recombination site.

19. A kit comprising the set of three frame adapters of claim 17.

20. The kit of claim 19, further comprising a primer comprising a poly (T) sequence.


SEQUENCE LISTING (9 sequences)

SEQ ID NO: 1 - DNA, 60 bp, Artificial
  Biotin-attB2-d(T)25 VN primer
  misc_feature (60)..(60): n is a, c, g, or t
acaactttgtacaagaaagttgggtgcggccgctttttttttttttttttttttttttvn

SEQ ID NO: 2 - DNA, 32 bp, Artificial
  adapter oligomer RF alpha long strand
tcgtcggggacaactttgtacaaaaaagttgg

SEQ ID NO: 3 - DNA, 27 bp, Artificial
  adapter oligomer RF alpha short strand
ccaacttttttgtacaaagttgtcccc

SEQ ID NO: 4 - DNA, 33 bp, Artificial
  adapter oligomer RF beta long strand
tcgtcggggacaactttgtacaaaaaagttgga

SEQ ID NO: 5 - DNA, 28 bp, Artificial
  adapter oligomer RF beta short strand
tccaacttttttgtacaaagttgtcccc

SEQ ID NO: 6 - DNA, 34 bp, Artificial
  adapter oligomer RF gamma long strand
tcgtcggggacaactttgtacaaaaaagttggaa

SEQ ID NO: 7 - DNA, 29 bp, Artificial
  adapter oligomer RF gamma short strand
ttccaacttttttgtacaaagttgtcccc

SEQ ID NO: 8 - DNA, 21 bp, Artificial
  forward sequencing/PCR primer
tataacgcgtttggaatcact

SEQ ID NO: 9 - DNA, 23 bp, Artificial
  reverse sequencing/PCR primer
agccgacaaccttgattggagac